(19) United States
(12) Patent Application Publication
(10) Pub. No.: US 2009/0104657 A1
(43) Pub. Date: Apr. 23, 2009

(54) Lipase Production Method

(75) Inventors: Anke Beselin, Mulheim (DE); Frank Rosenau, Ratingen (DE); Karl-Erich Jaeger, Dusseldorf (DE); Michael Breuer, Darmstadt (DE); Bernhard Hauer, Fussgongeim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/922,176

(22) PCT Filed: Jun. 13, 2006
(86) PCT No.: PCT/EP2006/005668

§ 371 Date: Dec. 13, 2007

(30) Foreign Application Priority Data
Jun. 15, 2005 (DE) 10 2005 027 685.7

(51) Int. Cl. C12P 21/00; C07K 14/00; C12N 9/48; C07H 21/00; C12N 15/63; C12N 1/21

(52) U.S. Cl. 435/69.1; 530/350; 435/212; 536/23.2; 435/320.1; 435/252.3

(57) ABSTRACT

The invention relates to novel proteins, especially with protease activity, which promote the production of extracellular lipases by bacteria, especially of the genus Burkholderia, nucleic acid sequences encoding them, expression constructs containing them, hosts transformed with them, methods for the production of the proteins with protease activity and methods for the production of lipases, especially by bacteria of the genus Burkholderia.

The invention relates to novel proteins, in particular with protease activity, which promote the production of extracellular lipases by bacteria; especially of the genus Burkholderia, nucleic acid sequences which code for them, expression constructs containing them, hosts transformed with them, methods for the production of the proteins with protease activity as well as methods of production of lipases, in particular by bacteria of the genus Burkholderia.

### BACKGROUND OF THE INVENTION

Proteases are of wide occurrence in nature and have numerous physiological functions. They are degradative enzymes, which catalyze the cleavage of peptide bonds. Extracellular proteases split large proteins into smaller molecules for subsequent absorption, whereas intracellular proteases play an important role in the regulation of metabolism. Their proteolytic activity includes, for example, the activation of zymogenic forms of enzymes by limited proteolysis, the processing and transport of secretory proteins through membranes or the regulation of gene expression by degradation of regulatory proteins or modification of ribosomal proteins [16].

In the year 2000, various proteases were identified in the genome of Pseudomonas aeruginosa, which displayed significant homology with the proteases DegP, Prc, protease III and SohB of Escherichia coli. After inactivation of the corresponding genes in the genome of P. aeruginosa, three mutants displayed higher lipase activity, whereas one mutant had lower lipase activity in the supernatant. Further experiments showed that the gene expression of the LipAB operon was not affected, which confirmed that these proteases influence the folding and/or secretion of the lipase LipA in P. aeruginosa [24].

Burkholderia glumae is a plant pathogen, which causes husk rot and mildew on the shoots and panicles of rice plants. Like many other bacteria, B. glumae produces an extracellular lipase (EC 3.1.1.3), which proved useful in a number of various biotechnological applications [18].

Production of this lipase at high yield would therefore be desirable. In order to construct a suitable overexpressing strain it is necessary to identify potential bottlenecks in lipase production. These bottlenecks are to be expected at the level of gene expression, folding and secretion of the enzyme into the culture medium.

The problem to be solved by the invention was therefore to find a way of improving extracellular lipase production by B. glumae.

### SUMMARY OF THE INVENTION

The above problem was solved by supplying a protein, comprising protease activity, which exerts a beneficial effect on the expression and/or folding and secretion of the lipase lipA in B. glumae.

According to the invention, first a cosmid library of B. glumae PG1 was constructed using the vector pLAFR3 with broad host specificity [20]. After screening about 2500 clones, 15 cosmids were identified which have an influence on lipase production. The corresponding DNA fragments of 2 cosmids were subcloned into a vector with broad host specificity, obtaining 5 different plasmids. Expression of these plasmids in B. glumae produced a clone that exhibited increased lipase activity. After DNA sequencing of the corresponding DNA fragment, the open reading frames (ORFs) were identified using the Open-Reading-Frame search program (ORF Finder) of the National Center of Biotechnology Information (NCBI). The longest ORF comprised 540 base pairs and the amino acid sequence derived from it contained a conserved domain, which is found in the ThiJ/Pfpl-G family. This family comprises various proteins, such as proteases, transcription regulators, RNA-binding proteins or chaperones [3]. It is therefore assumed that this ORF codes for an as yet uncharacterized protein, which constitutes a cytoplasmic protease that has a beneficial effect on the expression and/or folding and secretion of the lipase lipA in B. glumae.

### DESCRIPTION OF THE FIGURES

The figures show

FIG. 1 a multiple sequence alignment of an ORF that codes for a protease from Burkholderia glumae with proteins from databases. First the amino acid sequence of the protease was submitted to a sequence similarity search using the WU-BLAST2 program from EBL-EMBI with a BLOSUM62 matrix. The proteins with high similarity to the protease were then selected for constructing a multiple sequence alignment using Db-Clustal from EBL-EMBI. Residues within a column, which are identical in all sequences of the alignment, were marked with an asterisk. A colon stands for a conservative substitution. Semi-conservative substitutions are marked with a single dot. The protease according to the invention is shown in the first line. The next lines show: Q8XA99: Hypothetical protein yhbO from Escherichia coli. Q7CPQ5: Putative intracellular proteinase XHBO from Salmonella typhimurium. Q8XH07: Hypothetical protein STY3452 from S. typhimurium. Q5WER6: General stress protein GSP18 from Bacillus clausii. Q65MG4: YfkM from B. lichenformis. Q9K8l1: General stress protein from B. halodurans BH3025. Q370CX9: ThiJ/Pfpl-family protein from B. cereus (strain ATCC 10987) BCE0935. Q65FG2: Protease I, ThiJ/Pfpl-family protein from B. cereus. PFI_PYRHO: Protease I from Pyrococcus horikoshii OT3. PFPI_PYRFU: Protease I from Pyrococcus furiosus.

FIG. 2 the types of plasmids of plasmid constructs used according to the invention, namely: FIG. 2A the plasmid plTpro and FIG. 2B the plasmid pBBRpro.

FIG. 3 the relative lipolytic activity, observed on expression of various plasmids, which contained subcloned DNA from B. glumae PG1. The error bars show the standard deviation from 4 separate experiments. Lipase activity was determined spectrophotometrically with p-nitrophenylpalmitate as substrate and is shown as relative lipolytic activity relative to the wild-type strain [21]. The empty expression vector pBBR1 mcs served as control. Expression of the plasmid pBBRPG8/1, which contained the gene for the protease according to the invention, led to an increase in lipase activity by 20 to 30%.

FIG. 4 Restriction analysis of the plasmids pBBRPPG 5/1, 3, 7, 8/1 and 3 using the restriction enzymes XbaI and HincII. 1 μg DNA was isolated and was applied to 0.8% agarose gel. The DNA markers were obtained from Invitrogen.

FIG. 5 Homologous expression of the putative protease in B. glumae PG1 (a) and LU8093 (b). Expression was performed in four separate experiments using PG medium with 1% olive oil as inducer of lipase production. After 24 hours the cultures were harvested and OD₅₈₀ was determined. The extracellular lipase activity was determined spectrophotometrically using p-nitrophenylpalmitate as substrate (according to Winkler et al., 1978 [31]). The relative lipolytic activities were calculated by correlation of Δ410/min with OD₅₈₀. As a control, the strains were also transformed with the empty vector pBBR1 mcs and tested at the same time.

FIG. 6 Overexpression of the protease according to the invention in E. coli BL12DE3. Samples were taken after 0, 2 and 4 hours (T₀, T₂, T₄) and fractionated by 15% SDS-PAGE. The gel was stained with Coomassie Blue R-250. Lane 1: Protein standard M12 (Invitrogen), 2: T₀ BL21DE3 pET22b, 3: T₀ BL21DE3 pETpro, 4: T₂ BL21DE3 pET22b, 5: T₀ BL21DE3 pETpro, 6: T₄ BL21DE3 pET22b, 7: T₄ BL21DE3 pETpro.

FIG. 7 SDS-PAGE analysis for each purification step. Lane 1: Protein standard PageRuler (Fermentas) 2: 10 μl cellular lysate (1:5). 3: 10 μl percolation. 4: 10 μl wash fraction. 5: 10 μl eluate (1:13).

### DETAILED DESCRIPTION OF THE INVENTION

### 1. Preferred Embodiments

A first object of the invention relates to proteins, characterized by at least one of the following properties:
a) an amino acid sequence, comprising the consensus sequence:

| | |
|---|---|
| AICHGP | (SEQ ID NO: 7) |

b) no Helix-Turn-Helix (HTH) DNA-binding domain;
c) a molecular weight of about 20-22 kDa, determined by SDS-PAGE in denaturing conditions.

For example, one or two or three of the characteristics a), b) and c) can be present in any combination, and in particular these characteristics can occur simultaneously.

Furthermore, the protein according to the invention can have protease activity.

Preferably the proteins according to the invention possess a pl value in the range from 5.4 to 5.5. They can be obtained from bacteria of the genus Burkholderia, in particular Burkholderia glumae.

The proteins according to the invention are further characterized in that, after expression in a bacterial host that produces extracellular lipase, such as bacteria of the genus Burkholderia, in particular in Burkholderia glumae, they increase the extracellular lipolytic activity, determined in standard conditions. The extracellular lipolytic activity is increased by at least about 1%, e.g. about 5 to 200% or 10 to 100% or 20 to 50%, compared to the baseline value.

In particular, the protein according to the invention comprises an amino acid sequence according to SEQ ID NO: 2; or an amino acid sequence derived from it with at least 80% sequence homology.

The invention also relates to proteins, encoded by a nucleic acid, comprising SEQ ID NO: 1 or a sequence derived from it with at least 80% sequence homology, as well as these coding nucleic acid sequences themselves.

A further object of the invention relates to expression vectors, comprising, under the genetic control of at least one regulatory nucleic acid sequence, a nucleic acid sequence coding for a protein with protease activity according to the above definition.

A further object of the invention is a recombinant microorganism, genetically modified with at least one expression vector as defined above.

The invention further relates to a method of production of a protein with protease activity as defined above, in which a recombinant microorganism is cultivated in conditions expressing this protein and the protein that forms is isolated.

The invention also relates to a method of producing a preferably extracellular lipase (E.C. 3.1.1.3), in which a host that is capable of producing this lipase is caused to express a functional protein with protease activity as defined above and is caused to express lipase simultaneously or at a different time and the lipase that forms is isolated. In particular, the host is a bacterium of the genus Burkholderia, in particular Burkholderia glumae.

The lipase produced comprises, according to a preferred variant, an amino acid sequence according to SEQ ID NO: 6 or an amino acid sequence derived from it with at least 80% sequence homology, or is encoded by a nucleic acid sequence, comprising a sequence according to SEQ ID NO: 5 or a nucleic acid sequence derived from it with at least 80% sequence homology.

### 2. Explanation of General Terms

A “protein with protease activity” denotes, in at least one of the test methods described herein, the enzymatic activity of a protease (proteolytic activity), for example, but not limited to this, a proteolytic activity determined with at least one suitable protease substrate. We may mention, as non-limiting examples, aminopeptidase substrates, such as lysine-β-Na, arginine-β-Na, L-alanine-β-Na, glutamate(βNa)—OH. The enzymatic activity of the protein need not, however, be limited to proteolytic activity.

An “HTH binding domain” means a helix-turn-helix DNA-binding motif in protein sequences, as described by Dodd et al. (1990) in [26], which is expressly referred to herein.

“Extracellular lipases” in the sense of the invention are in particular those of Enzyme class E.C. 3.1.1.3. Preferably they are produced by bacteria of the genus Burkholderia, in particular by Burkholderia glumae, such as in particular the lipase lipA. Such lipases find application in particular in biotechnological processes, as described for example in Breuer et al. (2004) [27], Jaeger et al. (2002) [28] and Schmid et al. (2001) [29], which are expressly referred to herein.

“Extracellular lipolytic activity, determined in standard conditions”, means the activity determined using the standard spectrophotometric method of determination with p-nitrophenylpalmitate as substrate according to Winkler et al. [31] (final substrate concentration 0.8 mM; 37° C.; in Soerensen phosphate buffer), as described in the experimental part.

A “derived” sequence, e.g. a derived amino acid or nucleic acid sequence, means, according to the invention, unless stated otherwise, a sequence that has identity of at least 80% or at least 90%, in particular 91%, 92%, 93%, 94%, 95%, 96%, 97%, 98% and 99%, with the starting sequence.

“Identity” between two nucleic acids means identity of the nucleotides, in each case over the entire length of the nucleic acid, in particular the identity calculated by means of the Vector NTI Suite 7.1 program of the company Informax (USA) employing the Clustal Method (Higgins D G, Sharp P M. Fast and sensitive multiple sequence alignments on a microcomputer. Comput Appl. Biosci. 1989 April; 5(2):151-1) with the following settings:

Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise Alignment Parameter:

| | |
|---|---|
| FAST algorithm on | 1 |
| K-tuple size | |
| Gap penalty | 3 |
| Window size | 5 |
| Number of best diagonals | 5 |

### 3. Other Embodiments of the Invention

### 3.1 Proteins According to the Invention

The present invention is not limited to the concretely disclosed proteins or enzymes with protease activity, but also extends to functional equivalents thereof.

“Functional equivalents” or analogs of the concretely disclosed enzymes are, within the scope of the present invention, various polypeptides thereof, which moreover possess the desired biological activity, e.g. proteolytic activity.

For example, “functional equivalents” means enzymes which, in the test used for proteolytic activity, display at least 20%, preferably 50%, especially preferably 75%, quite especially preferably 90% of the activity of an enzyme, comprising an amino acid sequence according to SEQ ID NO: 2. Functional equivalents are moreover preferably stable between pH 4 to 10 and advantageously possess an optimum pH in the range of pH 5 to 8 as well as an optimum temperature in the range of 20° C. to 80° C.

The proteolytic activity can be demonstrated by means of various known tests for determination of proteolytic activity. Without being limited to it, we may mention a test using skimmed-milk agar plates, which contained per liter: 30 g skimmed-milk powder, 5 g yeast extract, 10 g NaCl, 10 g Trypton, 15 g agar and 1 mM IPTG. Individual colonies of the expression cultures or 5 μl of one of each purification step were applied to agar plates and incubated overnight at 30° C. (B. glumae) or 37° C. (E. coli). The proteolytic activity can be detected from the formation of haloes around the colonies or samples.

“Functional equivalents”, according to the invention, also means in particular mutants, which, in at least one sequence position of the amino acid sequences stated above, have an amino acid that is different from that concretely stated, but nevertheless possess one of the aforementioned biological activities. “Functional equivalents” thus comprise the mutants obtainable by one or more amino acid additions, substitutions, deletions and/or inversions, where the stated changes can occur in any sequence position, provided they lead to a mutant with the profile of properties according to the invention. Functional equivalence is in particular also provided if the reactivity patterns coincide qualitatively between the mutant and the unchanged polypeptide, i.e. if for example the same substrates are converted at a different rate. Examples of suitable amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxy groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent enzymes can be determined on the basis of the concrete parameters of the invention.

“Functional equivalents” also comprise fragments, preferably individual domains or sequence motifs, of the polypeptides according to the invention, which for example display the desired biological function.

“Functional equivalents” are, moreover, fusion proteins, which have one of the polypeptide sequences stated above or functional equivalents derived therefrom and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” that are also included according to the invention are homologs of the concretely disclosed proteins. These possess at least 60%, preferably at least 75% in particular at least 85%, e.g. 90, 91, 92, 93, 94, 95, 96, 97, 98 or 99%, homology with the concretely disclosed amino acid sequences, calculated according to the algorithm of Pearson and Lipman, Proc. Natl. Acad, Sci. (USA) 85(8), 1988, 2444-2448. A percentage homology of a homologous polypeptide according to the invention means in particular the percentage identity of the amino acid residues relative to the total length of one of the amino acid sequences concretely described herein.

In the case of a possible protein glycosylation, “functional equivalents” according to the invention comprise proteins of the type designated above in deglycosylated or glycosylated form as well as modified forms that can be obtained by altering the glycosylation pattern.

Homologs of the proteins or polypeptides according to the invention can be produced by mutagenesis, e.g. by point mutation, lengthening or shortening of the protein.

Homologs of the proteins according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologs from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologs according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologs (Arkin and Yourvan (1992) PNAS 89:7811-7815; Delgrave et al. (1993) Protein Engineering 6(3):327-331).

### 3.2 Coding Nucleic Acid Sequences

The invention also relates to nucleic acid sequences that code for an enzyme with proteinase activity. Nucleic acid sequences comprising a sequence according to SEQ ID NO: 1; or a nucleic acid sequence derived from the amino acid sequence according to SEQ ID NO.: 2 are preferred.

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), Molecular Cloning: A laboratory manual, Cold Spring Harbor Laboratory Press.

The invention also relates to nucleic acid sequences (single-stranded and double-stranded DNA and RNA sequences, e.g. cDNA and mRNA), coding for one of the above polypeptides and their functional equivalents, which can be obtained for example using artificial nucleotide analogs.

The invention relates both to isolated nucleic acid molecules, which code for polypeptides or proteins according to the invention or biologically active segments thereof, and to nucleic acid fragments, which can be used for example as hybridization probes or primers for identifying or amplifying coding nucleic acids according to the invention.

The nucleic acid molecules according to the invention can in addition contain untranslated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (see below) on at least about 12, preferably at least about 25, for example about 40, 50 or 75 successive nucleotides of a sense strand of a nucleic acid sequence according to the invention or of a corresponding antisense strand.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

A nucleic acid molecule according to the invention can be isolated by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention, such as SEQ ID No: 1 or derivatives thereof, homologs or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These standard conditions vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA: RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids the hybridization conditions are advantageously 0.1×SSC and temperatures between about 30° C. to 55° C., preferably between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., “Molecular Cloning”, Cold Spring Harbor Laboratory, 1989, and can be calculated using formulas that are known by a person skilled in the art, for example depending on the length of the nucleic acids, the type of hybrids or the G+C content. A person skilled in the art can obtain further information on hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

“Hybridization” can in particular be carried out under stringent conditions. Such hybridization conditions are for example described in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions mean in particular: Incubation at 42° C. overnight in a solution consisting of 50% formamide, 5×SSC (750 mM NaCl, 75 mM tri-sodium citrate), 50 mM sodium phosphate (pH 7.6), 5×Denhardt Solution, 10% dextran sulfate and 20 g/ml denatured, sheared salmon sperm DNA, followed by washing of the filters with 0.1×SSC at 65° C.

The invention also relates to derivatives of the concretely disclosed or derivable nucleic acid sequences.

Thus, further nucleic acid sequences according to the invention can be derived e.g. from SEQ ID NO: 1 and can differ from it by addition, substitution, insertion or deletion of individual or several nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism, as well as naturally occurring variants, e.g. splicing variants or allelic variants, thereof.

It also relates to sequences that can be obtained by conservative nucleotide substitutions (i.e. the amino acid in question is replaced by an amino acid of the same charge, size, polarity and/or solubility).

The invention also relates to the molecules derived from the concretely disclosed nucleic acids by sequence polymorphisms. These genetic polymorphisms can exist between individuals within a population owing to natural variation. These natural variations usually produce a variance of 1 to 5% in the nucleotide sequence of a gene.

Derivatives of the nucleic acid sequence with the sequence SEQ ID NO: 1 according to the invention mean for example allelic variants, having at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

Furthermore, derivatives are also to be understood to be homologs of the nucleic acid sequences according to the invention, in particular of SEQ ID NO: 1, for example fungal or bacterial homologs, shortened sequences, single-stranded DNA or RNA of the coding and noncoding DNA sequence. For example, homologs of SEQ ID NO: 1 have, at the DNA level, a homology of at least 40%, preferably of at least 60%, especially preferably of at least 70%, quite especially preferably of at least 80% over the entire DNA region given in SEQ ID NO: 1.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 3.3 Constructs According to the Invention

The invention also relates to expression constructs, containing, under the genetic control of regulatory nucleic acid sequences, a nucleic acid sequence coding for a polypeptide according to the invention; as well as vectors comprising at least one of these expression constructs.

“Expression unit” means, according to the invention, a nucleic acid with expression activity, which comprises a promoter as defined herein and, after functional association with a nucleic acid that is to be expressed or a gene, regulates the expression, i.e. the transcription and the translation of this nucleic acid or of this gene. In this context, therefore, it is also called a “regulatory nucleic acid sequence”. In addition to the promoter, other regulatory elements may be present, e.g. enhancers.

“Expression cassette” or “expression construct” means, according to the invention, an expression unit, which is functionally associated with the nucleic acid that is to be expressed or the gene that is to be expressed. In contrast to an expression unit, an expression cassette thus comprises not only nucleic acid sequences which regulate transcription and translation, but also the nucleic acid sequences which should be expressed as protein as a result of the transcription and translation.

The terms “expression” or “overexpression” describe, in the context of the invention, the production or increase of intracellular activity of one or more enzymes in a microorganism, which are encoded by the corresponding DNA. For this, it is possible for example to insert a gene in an organism, replace an existing gene by another gene, increase the number of copies of the gene or genes, use a strong promoter or use a gene that codes for a corresponding enzyme with a high activity, and optionally these measures can be combined.

Preferably such constructs according to the invention comprise a promoter 5′-upstream from the respective coding sequence, and a terminator sequence 3′-downstream, and optionally further usual regulatory elements, in each case functionally associated with the coding sequence.

A “promotor”, a “nucleic acid with promotor activity” or a “promotor sequence” mean, according to the invention, a nucleic acid which, functionally associated with a nucleic acid that is to be transcribed, regulates the transcription of this nucleic acid.

“Functional” or “operative” association means, in this context, for example the sequential arrangement of one of the nucleic acids with promoter activity and of a nucleic acid sequence that is to be transcribed and optionally further regulatory elements, for example nucleic acid sequences that enable the transcription of nucleic acids, and for example a terminator, in such a way that each of the regulatory elements can fulfill its function in the transcription of the nucleic acid sequence. This does not necessarily require a direct association in the chemical sense. Genetic control sequences, such as enhancer sequences, can also exert their function on the target sequence from more remote positions or even from other DNA molecules. Arrangements are preferred in which the nucleic acid sequence that is to be transcribed is positioned behind (i.e. at the 3′ end) the promoter sequence, so that the two sequences are bound covalently to one another. The distance between the promoter sequence and the nucleic acid sequence that is to be expressed transgenically can be less than 200 bp (base pairs), or less than 100 bp or less than 50 bp.

Apart from promoters and terminators, examples of other regulatory elements that may be mentioned are targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, Calif. (1990).

Nucleic acid constructs according to the invention comprise in particular sequence SEQ ID NO: 1 or derivatives and homologs thereof, as well as the nucleic acid sequences that can be derived from SEQ ID NO: 2, which are advantageously associated operatively or functionally with one or more regulating signals for controlling, e.g. increasing, gene expression.

In addition to these regulatory sequences, the natural regulation of these sequences can still be present in front of the actual structural genes and optionally can have been altered genetically, so that natural regulation is switched off and the expression of the genes has been increased. The nucleic acid construct can also be of a simpler design, i.e. without any additional regulatory signals being inserted in front of the coding sequence (e.g. SEQ ID NO: 1 or its homologs) and without removing the natural promoter with its regulation. Instead, the natural regulatory sequence is silenced so that regulation no longer takes place and gene expression is increased.

A preferred nucleic acid construct advantageously also contains one or more of the aforementioned enhancer sequences, functionally associated with the promoter, which permit increased expression of the nucleic acid sequence. Additional advantageous sequences, such as other regulatory elements or terminators, can also be inserted at the 3′ end of the DNA sequences. One or more copies of the nucleic acids according to the invention can be contained in the construct. The construct can also contain other markers, such as antibiotic resistances or auxotrophy-complementing genes, optionally for selection on the construct.

Examples of suitable regulatory sequences are contained in promoters such as cos-, tac-, trp-, tet-, trp-tet-, lpp-, lac-, lpp-lac-, lacl^{q-}, T7-, T5-, T3-, gal-, trc-, ara-, rhaP (rhaP_{BAD})SP6-, lambda-P_{R}- or in the lambda-P_{L} promoter, which find application advantageously in Gram-negative bacteria. Other advantageous regulatory sequences are contained for example in the Gram-positive promoters amy and SPO2, in the yeast or fungal promoters ADC1, MFalpha, AC, P-60, CYC1, GAPDH, TEF, rp28, ADH. Artificial promoters can also be used for regulation.

For expression, the nucleic acid construct is inserted in a host organism advantageously in a vector, for example a plasmid or a phage which permits optimum expression of the genes in the host. In addition to plasmids and phages, vectors are also to be understood as meaning all other vectors known to a person skilled in the art, e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent a further embodiment of the invention.

Suitable plasmids are, for example in E. coli, pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl; in streptomyces plJ101, plJ364, plJ702 or plJ361; in bacillus pUB110, pC194 or pBD214; in corynebacterium pSA77 or pAJ667; in fungi pALS1, plL2 or pBB116; in yeasts 2alphaM, pAG-1, YEp6, YEp13 or pEMBLYe23 or in plants pLGV23, pGHlac⁺, pBIN19, pAK2004 or pDH51. The aforementioned plasmids represent a small selection of the possible plasmids. Other plasmids are well known to a person skilled in the art and will be found for example in the book Cloning Vectors (Eds. Pouwels P. H. et al. Elsevier, Amsterdam-New York-Oxford, 1985, ISBN 0 444 904018).

In a further embodiment of the vector, the vector containing the nucleic acid construct according to the invention or the nucleic acid according to the invention can be inserted advantageously in the form of a linear DNA in the microorganisms and integrated into the genome of the host organism through heterologous or homologous recombination. This linear DNA can comprise a linearized vector such as plasmid or just the nucleic acid construct or the nucleic acid according to the invention.

For optimum expression of heterologous genes in organisms, it is advantageous to alter the nucleic acid sequences in accordance with the specific codon usage employed in the organism. The codon usage can easily be determined on the basis of computer evaluations of other, known genes of the organism in question.

The production of an expression cassette according to the invention is based on fusion of a suitable promoter with a suitable coding nucleotide sequence and a terminator signal or polyadenylation signal. Common recombination and cloning techniques are used for this, as described for example in T. Maniatis, E. F. Fritsch and J. Sambrook, Molecular Cloning: A Laboratory Manual, Cold Spring Harbor Laboratory, Cold Spring Harbor, N.Y. (1989) as well as in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is inserted advantageously in a host-specific vector for expression in a suitable host organism, to permit optimum expression of the genes in the host. Vectors are well known to a person skilled in the art and will be found for example in “Cloning Vectors” (Pouwels P. H. et al., Publ. Elsevier, Amsterdam-New York-Oxford, 1985).

### 3.4 Hosts that can be Used According to the Invention

Depending on the context, the term “microorganism” means the starting microorganism (wild-type) or a genetically modified microorganism according to the invention, or both.

The term “wild-type” means, according to the invention, the corresponding starting microorganism, and need not necessarily correspond to a naturally occurring organism.

By means of the vectors according to the invention, recombinant microorganisms can be produced, which have been transformed for example with at least one vector according to the invention and can be used for production of the polypeptides according to the invention. Advantageously, the recombinant constructs according to the invention, described above, are inserted in a suitable host system and expressed. Preferably, common cloning and transfection methods that are familiar to a person skilled in the art are used, for example co-precipitation, protoplast fusion, electroporation, retroviral transfection and the like, in order to secure expression of the stated nucleic acids in the respective expression system. Suitable systems are described for example in Current Protocols in Molecular Biology, F. Ausubel et al., Publ. Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, N.Y., 1989.

In principle, all prokaryotic or eukaryotic organisms can be considered as recombinant host organisms for the nucleic acid according to the invention or the nucleic acid construct. Microorganisms such as bacteria, fungi or yeasts are used advantageously as host organisms. It is advantageous to use Gram-positive or Gram-negative bacteria, preferably bacteria of the families Enterobacteriaceae, Pseudomonadaceae, Rhizobiaceae, Streptomycetaceae or Nocardiaceae, especially preferably bacteria of the genera Escherichia, Pseudomonas, Streptomyces, Nocardia, Burkholderia, Salmonella, Agrobacterium or Rhodococcus. The genus and species Escherichia coli is quite especially preferred. Other advantageous bacteria will also be found in the following group: alpha-proteobacteria, beta-proteobacteria or gamma-proteobacteria.

The host organism or host organisms according to the invention then preferably contain at least one of the nucleic acid sequences, nucleic acid constructs or vectors described in this invention, which code for an enzyme with protease activity according to the above definition.

The organisms used in the method according to the invention are grown or bred in a manner familiar to a person skilled in the art, depending on the host organism. As a rule, microorganisms are grown in a liquid medium, which contains a source of carbon, generally in the form of sugars, a source of nitrogen generally in the form of organic sources of nitrogen such as yeast extract or salts such as ammonium sulfate, trace elements such as iron, manganese and magnesium salts and optionally vitamins, at temperatures between 0° C. and 100° C., preferably between 10° C. to 60° C. with oxygen aeration. The pH of the liquid nutrient medium can be maintained at a fixed value, i.e. regulated or not regulated during growing. Growing can be carried out batchwise, semi-batchwise or continuously. Nutrients can be supplied at the start of fermentation or can be supplied subsequently, either semi-continuously or continuously. The ketone can be added directly during growing, or advantageously after growing. The enzymes can be isolated from the organisms by the method described in the examples or can be used as raw extract for the reaction.

Hosts that can be used according to the invention are in particular bacteria of the genus Burkholderia, in particular of the species Burkholderia glumae. The generally available strain DSM No: 9512 of Burkholderia glumae (synonym: Pseudomonas glumae) may be mentioned as a non-limiting example.

### 3.5 Recombinant Production of the Protease

The invention also relates to methods for recombinant production of polypeptides according to the invention or functional, biologically active fragments thereof, by cultivating a polypeptide-producing microorganism, if necessary inducing expression of the polypeptides and isolating them from the culture. The polypeptides can also be produced on an industrial scale in this way, if so desired.

The microorganisms produced according to the invention can be cultivated continuously or discontinuously in the batch process or in the fed batch or repeated fed batch process. A review of known methods of cultivation will be found in the textbook by Chmiel (Bioprocesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium that is to be used must satisfy the requirements of the particular strains in an appropriate manner. Descriptions of culture media for various microorganisms are given in the handbook “Manual of Methods for General Bacteriology” of the American Society for Bacteriology (Washington D.C., USA, 1981).

These media that can be used according to the invention generally comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose. Sugars can also be added to the media via complex compounds, such as molasses, or other by-products from sugar refining. It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soybean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, but also organic sulfur compounds, such as mercaptans and thiols, can be used as sources of sulfur.

Phosphoric acid, potassium dihydrogenphosphate or dipotassium hydrogenphosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (Publ. P. M. Rhodes, P. F. Stanbury, IRL Press (1997) p. 53-73, ISBN 0 19 963577 3). Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

The temperature of the culture is normally between 15° C. and 45° C., preferably 25° C. to 40° C. and can be kept constant or can be varied during the experiment. The pH value of the medium should be in the range from 5 to 8.5, preferably around 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions. The temperature of the culture is normally from 20° C. to 45° C. Culture is continued until a maximum of the desired product has formed. This is normally achieved within 10 hours to 160 hours.

The fermentation broth is then processed further. Depending on the requirements, the biomass can be removed completely or partially from the fermentation broth by separation techniques, e.g. centrifugation, filtration, decanting or a combination of these methods, or can be left in the fermentation broth completely.

If the polypeptides are not secreted into the culture medium, the cells can be disrupted and the product can be obtained from the lysate by known techniques for isolating proteins. The cells can be disrupted optionally by high-frequency ultrasound, by high pressure, e.g. in a French pressure cell, by osmolysis, by the action of detergents, lytic enzymes or organic solvents, by means of homogenizers or by a combination of several of the methods listed.

The polypeptides can be purified using known chromatographic methods, such as molecular sieve chromatography (gel filtration), Q-Sepharose chromatography, ion-exchange chromatography and hydrophobic chromatography, and by other usual methods such as ultrafiltration, crystallization, salting-out, dialysis and native gel electrophoresis. Suitable methods are described for example in Cooper, F. G., Biochemische Arbeitsmethoden, Verlag Walter de Gruyter, Berlin, N.Y. or in Scopes, R., Protein Purification, Springer Verlag, New York, Heidelberg, Berlin.

For isolating the recombinant protein it may be advantageous to use vector systems or oligonucleotides, which extend the cDNA by defined nucleotide sequences and therefore code for modified polypeptides or fusion proteins, which can be used e.g. for simpler purification. Suitable modifications of this kind are for example so-called “tags” which function as anchors, e.g. the modification known as the hexa-histidine anchor, or epitopes that can be recognized as antigens by antibodies (described for example in Harlow, E. and Lane, D., 1988, Antibodies: A Laboratory Manual. Cold Spring Harbor (N.Y.) Press). These anchors can provide adhesion of the proteins to a solid support, e.g. a polymer matrix, for example for packing a chromatographic column, or can be used on a microtiter plate or on some other support.

At the same time, these anchors can also be used for recognition of the proteins. For recognition of the proteins it is also possible to use ordinary markers, such as fluorescent dyes and enzyme markers which form a detectable reaction product after reaction with a substrate, or radioactive markers, alone or in combination with the anchors for derivatization of the proteins.

### 3.6 Execution of the Method According to the Invention for the Production of Lipases with B. glumae

Essentially, the lipase is produced by fermentation of Burkholderia glumae. The organism is grown in a special production medium. The lipase is secreted by Burkholderia glumae in active form into the medium. At the end of fermentation, the cells are separated from the medium by decanting/centrifugation. The resulting residue can be dried, to obtain lipase as a dry formulation. The lipase can also be obtained in ultrapure form from the medium by suitable chromatographic methods. Finally, direct adsorption from the medium on suitable supports by immobilization is also possible. Standard methods that are familiar to a protein specialist are employed for this.

Otherwise the microbial production and isolation of extracellular lipase are carried out as for the protease (cf. above).

The lipases that are more easily available according to the invention can be used for example for the production of fine chemicals. In particular, they are employed for obtaining optically active compounds by resolution of racemates. However, use as catalysts for enantioselective synthesis is also conceivable. Finally, the enzyme can find application not only on account of its stereoselectivity, but also on account of its regioselectivity. Possible areas of application include the modification of polymers or low-molecular compounds, in which ester bonds are split or joined regioselectively.

The following examples only serve to illustrate the invention. The numerous possible variations that are obvious to a person skilled in the art also fall within the scope of the invention.

Experimental Part

The cloning steps carried out within the scope of the present invention, such as restriction cleavage, agarose gel electrophoresis, purification of DNA fragments, transfer of nucleic acids on nitrocellulose and nylon membranes, joining of DNA fragments, transformation of E. coli cells, growing of bacteria, replication of phages and sequencing of recombinant DNA were carried out as described in Sambrook et al. (1989) op. cit.

1. Description of the Experiments

a) Bacterial Strains and Growing Conditions

E. coli DH5α was used for the routine cloning experiments. This strain was cultivated in Luria Bertani medium at 37° C. (LB medium (pH 7.0): 10 g/l NaCl, 10 g/l Bacto-Trypton, 5 g/l yeast extract; Sambrook J. et al. [17]. Ampicillin (100 μg/ml), tetracycline (25 μg/ml) or chloramphenicol (50 μg/ml) were used for conservation of the plasmid. The wild-type strain B. glumae PG1 (obtained from Jan Tommassen, Utrecht University, the Netherlands) and the production strain B. glumae LU8093 were used for the expression studies.

These strains were cultivated at 30° C. in PG medium, which contained per liter: 6 g (NH₄)₂SO₄, 3.5 g KH₂PO₄, 3.5 g K₂HPO₄, 0.02 g CaCl₂, 1 g MgSO₄·7H₂O and 2 g yeast extract, pH 6.5 [6]. For induction of lipase production, 1% (v/v) olive oil (Sigma) was added, and chloramphenicol (200 μg/ml) was added to the medium for conservation of the expression plasmid.

b) Production of Plasmid and Cosmid Constructs According to the Invention

General methods of DNA manipulation were carried out in accordance with Sambrook et al. [17] or according to manufacturers' instructions for DNA-modifying enzymes.

Construction of the genome library of B. glumae PG1 was carried out essentially as described by Staskawicz et al. [20]. After isolating genomic DNA from overnight cultures, the DNA was partially hydrolyzed using Sau3A. The DNA fragments were fractionated by electrophoresis on 0.5% agarose gel in standardized Tris-acetate-EDTA buffer and fragments larger than 15 kb were cut out and purified. The cosmid pLAFR3 (Staskawicz et al, [20]) was hydrolyzed in two different reactions with the restriction enzymes HindIII and EcoRI and then dephosphorylated. In a second step, the two restriction charges were digested with BamHI, in order to obtain Sau3A-compatible ends. Ligation of the genomic DNA and of the two DNA fragments of pLAFR3 was conducted overnight at 16° C. using T4 DNA-ligase. Finally the cosmids were packaged in vitro and transduced in E. coli JM101. Since the cosmid pLAFR3 permits blue/white screening, positive clones could be identified on LB agar plates, containing tetracycline (25 μg/ml), isopropyl-1-thio-β-D-galactopyranoside (IPTG, 250 μg/ml final concentration) and 5-bromo-4-chloro-3-indolyl-β-D-galactopyranoside (40-Gal, 40 μg/ml final concentration). In all, 9300 clones were selected and transferred to microtiter plates, which led to a 4-fold coverage of the B. glumae PG1 genome.

The DNA fragments of the cosmids pLAFRPG5 and pLAFRPG8 thus obtained exerted an influence on lipase production in B. glumae. The latter were subcloned by inserting the EcoRI/HindIII fragments in pBBR1mcs (Kovach, M. E. et al. [30]). This gave the plasmids pBBRPG 5/1, 5/3, 5/7, 8/1+8/3, which were submitted to DNA sequencing.

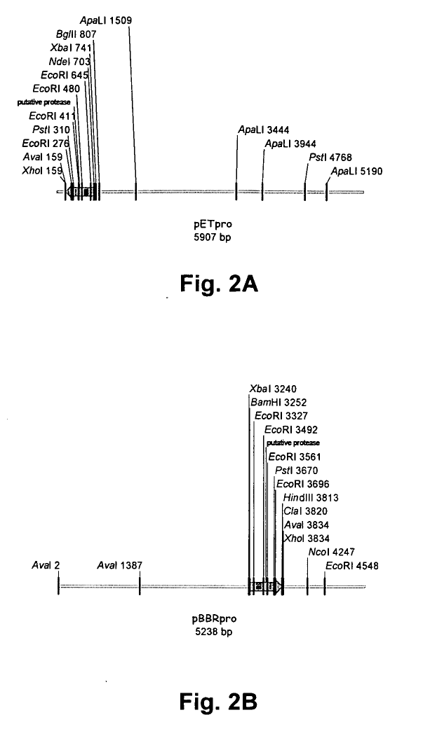

The DNA fragment that codes for the 179 amino acids of the protease according to the invention was amplified by the polymerase chain reaction using the genomic DNA from B. glumae PG1 as template. The oligonucleotide primer Proᵤₚ corresponding to the 5′ end of the amplified DNA (CTAGGATCCAATTCGCCTCATCCAATGCA) (SEQ ID NO: 8) was provided with a BamHI recognition sequence and the second primer Pro_{down} (CGCAAGCTTTCACGCGCCGGCCC) (SEQ ID NO: 9) was provided with a HindIII recognition sequence. The amplified DNA fragment with a size of 540 bp was cloned into the BamHI/HindIII site of the pBBR1mcs, expression of which was under the control of the lac promoter. The resulting plasmid with the designation pBBRpro (cf. FIG. 2B) was transferred into B. glumae by conjugation. For overexpression in E. coli, the PCR product was reamplified using the following primers: ProᵤₚN (GTACATATGCAGCAGCGTGGC) (SEQ ID NO: 10) with an NdeI recognition sequence and Pro_{down}X (ATCTCGAGTCACGCGCCGGCC) (SEQ ID NO: 11) with an XhoI recognition sequence. The DNA fragment was then cloned in the NdeI/HindIII position of pET22b (Novagen Inc., Madison, Wis., USA (1997)) and was thus under the control of the T7 promoter and had a C-terminal 6×His-tag, which makes purification possible by nickel-nitrilotriacetic acid metal-affinity chromatography. The resulting plasmid with the designation pETpro (cf. FIG. 2A) was transferred by transformation in E. coli.

c) Conjugative Transfer of Cosmids or Plasmids in B. glumae

The conjugative transfers of the cosmids were effected with E. coli JM101 as donor and E. coli pRK2013 as helper strain [6]. Conjugative transfers with the plasmids with broad host specificity pBBR1mcs and pBBRpro (produced from pBBR1mcs by cloning-in of the pro-gene via the cleavage sites HindIII and BamHI) were carried out biparentally using E. coli S17-1 as donor strain [19]. Overnight cultures of the recipients (B. glumae PG1 or LU8093) as well as logarithmic cultures of the donor and helper strains were centrifuged, washed and resuspended in 0.9% NaCl. 1-ml fractions of each culture were combined, centrifuged and resuspended in 50 μl 0.9% NaCl. The mixtures were applied dropwise on LB agar plates and dried. The plates were incubated overnight at 30° C. On the next day the spots were resuspended in 0.9% NaCl and plated on selective PG medium, containing tetracycline (50 μg/ml) and ampicillin (100 μm/ml) for cosmid screening or containing chloramphenicol (200 μg/ml) and ampicillin (100 μg/ml) for selection of the vectors with broad host specificity.

d) Expression Studies in the Homologous Host B. Glumae and Overexpression of the Recombinant Protein in the Heterologous Host E. coli

Expression of pBBRpro in the B. glumae strains PG1 and Lu8093 was carried out for 24 hours using PG medium +1% olive oil. The empty vector pBBR1mcs was expressed as control. After 24 hours, samples were taken, centrifuged and washed twice with 0.9% NaCl. Suitable dilutions of the pellets were used for determining the optical density (OD₅₈₀), whereas the supernatant was used for determination of lipase activity.

Overexpression of the protease according to the invention was carried out in E. coli using the T7 expression host E. coli BL21DE3. The strain was grown in LB medium, containing 100 μg/ml ampicillin for conservation of the overexpressing vector pETpro. Transformation of the vector pETpro in E. coli BL21DE3 was effected by heat shock (according to the RbCl₂ method of Hanahan, 1983 [32]) by addition of 1 μl of vector in 50 μl TMF buffer to a batch of transformation-competent cells, incubation on ice for 30 minutes, heat shock at 42° C. for 90 seconds, addition of 700 μl LB medium and incubation of the charge for 30 minutes at 37° C. Then the charge is plated on selective agar plates (LB agar with ampicillin 100 μg/ml).

After incubation for 2 hours at 37° C., expression of the T7 promoter was induced by addition of IPTG at a final concentration of 0.4 mM. Samples of the culture were taken after 2, 4 and 6 hours and analyzed by sodium dodecylsulfate (SDS)-polyacrylamide-gel electrophoresis.

e) Production of Cellular Extracts

The cells were adjusted to an optical density of 0.15, resuspended in 15 μl SDS buffer and incubated for 5 minutes at 95° C.

f) TCA Precipitation

1/10 volume of 1% sodium dodecylsulfate was added to the supernatant and incubated for 10 minutes at room temperature. The proteins were precipitated with 1/10 volume of 70% trichloroacetic acid (TCA) and incubation for 1 hour on ice. After centrifugation for 10 minutes (13000 rev/min, room temperature) the samples were washed twice with ice-cold 80% acetone and dried in a vacuum dryer. Finally the protein was resuspended in 15 μl SDS buffer and incubated for 5 minutes at 95° C.

g) SDS-Polyacrylamide Gel Electrophoresis

Sodium dodecylsulfate-polyacrylamide gel electrophoresis (SDS-PAGE) was carried out with 15% polyacrylamide gel according to Laemmli [13]. The proteins were stained with Coomassie Brilliant Blue R250.

h) Native Polyacrylamide Gel Electrophoresis

Native polyacrylamide gel electrophoresis was carried out using a 4 to 12% polyacrylamide gradient gel (Invitrogen) in accordance with the manufacturer's protocol. The proteins were stained with Coomassie Brilliant Blue G250.

i) Purification of the Protease According to the Invention

The protease according to the invention was purified by nickel-nitrilotriacetic acid metal-affinity chromatography. For this, 6 L of liquid culture of E. coli BL21 DE3 pETpro was grown at 37° C. until the optical density at 580 nm was 5.0. The cells were then induced by addition of 1 mM IPTG and grown for a further 4 hours at 37° C. After harvesting the cells by centrifugation (5000 rev/min, 15 minutes, 4° C.) the pellets were resuspended in 25 ml of ice-cold buffer (50 mM Kpi, 10 mM imidazole, pH 8.0). Cell disruption was effected by incubation with lysozyme (0.5 mg/ml, 30 minutes on ice), followed by sonication (10×30 seconds). To remove cell fragments, the sample was centrifuged again (14000 rev/min, 20 minutes, 4° C.) and filtered. The clear lysate was applied to a nickel-nitrilotriacetic acid column and washed with potassium phosphate buffer (50 mM Kpi, 20 mM imidazole, pH 8.0). After elution of the recombinant protein with 50 mM potassium phosphate buffer, containing 250 mM imidazole (pH 8.0) the corresponding fractions were combined and desalted using a Sephadex-G25 column. A volume of 50 μl of each of the purification steps was put aside for SDS-PAGE analysis and measurement of proteolytic enzyme activity. The protein concentrations were then determined using a Bradford assay [4] and using bovine serum albumin as the standard.

j) Enzyme Activity Assays

The lipolytic activity was determined on indicator plates that contained a tributyrin emulsion (7.5 ml tributyrin, 0.75 g gum arabic, distilled water to 15 ml) or alternatively spectrophotometrically with p-nitrophenylpalmitate (pNPP) as substrate, as described by Stuer et al. [21].

pNPP: 0.8 mM final concentration

Sørensen phosphate buffer: Solution A: 50 mM Na₂HPO₄×2H₂O
Solution B: 50 mM KH₂PO₄

Solutions A and B are mixed in the proportions 17:1

Substrate emulsion: Solution I: 207 mg sodium deoxycholate
100 mg gum arabic
90 ml Sørensen phosphate buffer

Solution II: 30 mg p-nitrophenylpalmitate
10 ml isopropanol

The substrate emulsion is freshly prepared before each activity determination and is used within one hour. The enzyme test is carried out at a constant 37°. 10-50 μl of a supernatant is added to 2 ml of preheated substrate emulsion and the change in OD₄₁₀ (ΔOD₄₁₀) is measured over a period of 5 minutes. The relative lipolytic activity is found from the correlation of ΔOD₄₁₀/min with the OD₅₈₀ of the cultures.

The proteolytic activity was determined using skimmed-milk agar plates, which contained per liter: 30 g skimmed-milk powder, 5 g yeast extract, 10 g NaCl, 10 g Trypton, 15 g agar and 1 mM IPTG. Individual colonies of the expression cultures or 5 μl of each of the purification steps were applied to agar plates and incubated overnight at 30° C. (B. glumae) or 37° C. (E. coli). Proteolytic activity can be detected by the formation of haloes around the colonies or samples.

k) Computer Analyses

The sequence alignments, ORF and database searches were carried out using standard software from the National Center for Biotechnology Information: Basic Local Alignment Search Tool (BLAST) [1, 2], Open Reading Frame-Finder (ORF-Finder) or European Bioinformatics Institute (EMBL-EBI): Washington University Basic Local Alignment Search Tool Version 2.0 (WU-BLAST2, db Clustal) [1, 210]. Prediction of a possible localization of the protein and computer analysis were carried out using the prediction program ProtParam tool [9], PSORT-B [8], SignalP 3.0 Server [14] from Expert Protein Analysis Systems (ExPASY) and the Helix-Turn-Helix-Prediction server from Pole BioInformatic, Lyons, Network Protein Sequence Analysis (PBIL, NPS, France) [5].

2. Test Results

a) Identification of Bottlenecks in Lipase Production of B. glumae

A genome library of B. glumae PG1 was constructed, as described above, using the cosmid pLAFR3 [19] with broad host specificity. After conjugation in B. glumae PG1 and LU8093 the cosmid database was screened using lipase indicator plates and lipase activity assays [21].

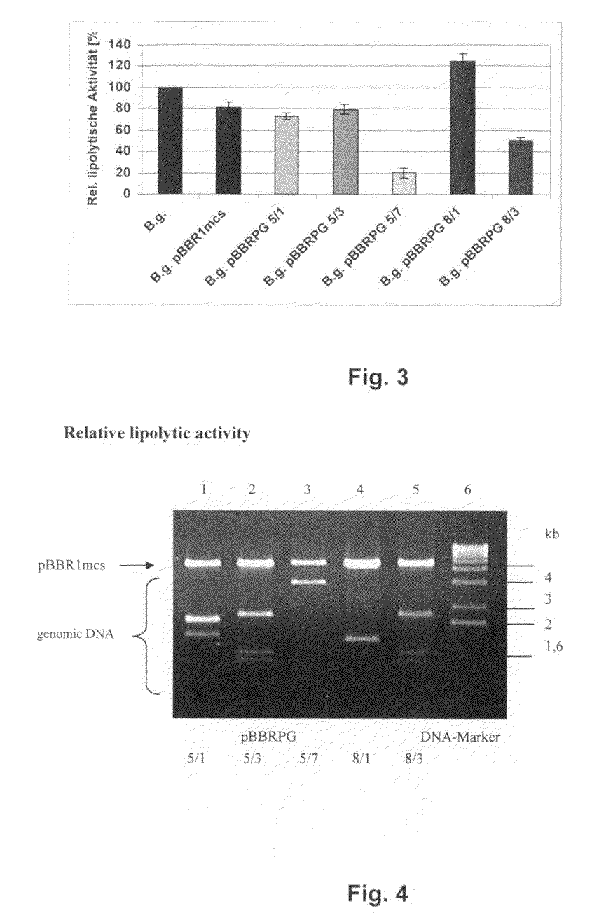

In a library of about 2500 clones, 15 cosmids having an influence on lipase production were identified. The corresponding DNA fragments from two of these cosmids were subcloned in pBBR1 mcs and identified by DNA sequencing. Further expression studies in B. glumae finally yielded a plasmid that led to a 20 to 30% increase in extracellular lipase activity (cf. FIG. 3).

b) Expression of a ThiJ/Pfpl Protein Leads to Increased Lipase Activity in B. glumae

Restriction analysis of the plasmid pBBRPG 8/1 (FIG. 3) and DNA sequencing of the inserts showed a DNA fragment size of about 1.2 kb (cf. FIG. 4, Lane 4).

Using the Open Reading Frame search program (ORF-Finder) of the National Center for Biotechnology Information (NCBI) three open reading frames (ORFs) were detected, the largest of which (540 bp) codes for a protein with a conserved domain, which is characteristic of proteins of the ThiiJ/Pfpl family. The other two ORFs comprise 444 or 348 bp and did not display any significant similarity with other annotated proteins of the database.

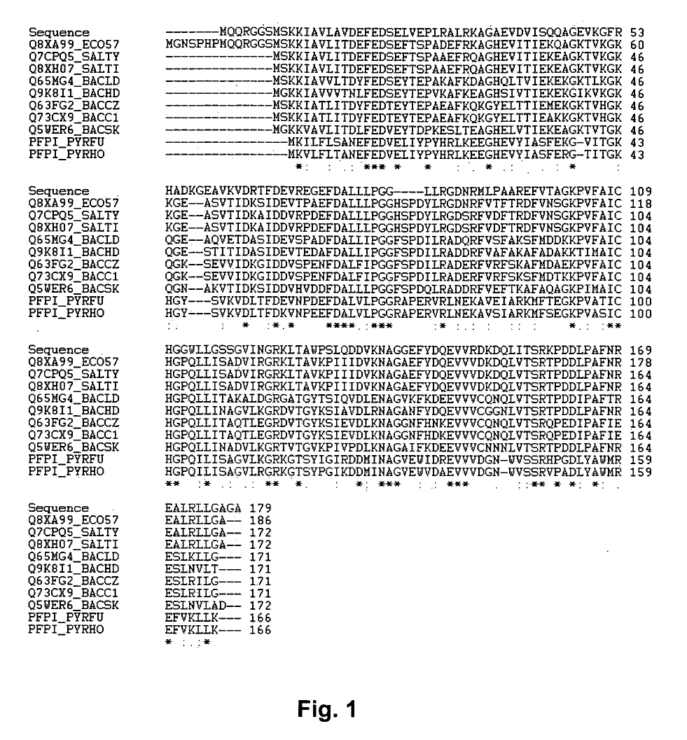

Comparison of the amino acid sequences with other proteins in the database using the WU-Blast2 program from EMBL-EBI showed significant similarities in several cases, in particular to putative intracellular proteases and stress proteins (cf. FIG. 1). For example, 69% of the amino acid sequence is similar to the intracellular protease YHBO from E. coli [15]. There was 49% similarity to the general stress protein YFKM from Bacillus clausii [22], 44% similarity to protease I from Bacillus cereus [11] and 44% agreement with the protease Pfpl and the protease PH1704 of the thermophilic bacteria Pyrococcus furiosus and P. horikoshii [25]. A recently published review of evolutionary and functional relations within the DJ-I/ThiJ/Pfpl superfamily shows that this superfamily comprises proteins with a variety of functions, such as proteases, transcription regulators, sigma-cross-reacting proteins, catalases etc. [3].

In addition to the human DJ-1 protein, which is involved in hereditary Parkinson's disease and the ThiJ or protease-related proteins from plants, there are also a number of bacterial proteins which have a DJ-1/ThiJ-like domain, for example the Large-Subunit-Catalases, containing a catalase domain and a DJ-1/ThiJ domain, or the transcription regulators of the AraC type. The latter can be identified from the presence of one or more Helix-Turn-Helix (HTH) motifs in the C-terminal part of the protein. The HTH motif presumably mediates DNA binding, whereas the ThiJ-like domain is an amidase [3]. An extraordinarily conserved composition (91.4% identity) is displayed by the family of the sigma-cross-reacting proteins, which distinguishes them from the adjacent families. These proteins have a distinct ElbB domain (ElbB=Enhancing lycopene biosynthesis protein 2 from E. coli) and display relatively moderate homology with the ThiJ proteins. Finally there are two groups which possess high similarity at the amino acid sequence level, which include the proteins of the above alignments (FIG. 1). The first group comprises the Pfpl proteases and contains the two proteases PfPl and PH1704 from the thermophilic bacteria Pyrococcus furiosus or Pyrococcus horikoshii. These proteases form hexameric ring structures and display ATP-independent protease activity, though only in oligomeric form. Owing to the presence of a cysteine residue (100) adjacent to a histidine residue, these proteins are classified as cysteine proteases. In the crystal structure of PH1704 the corresponding Cys100 residue is arranged in a nucleophilic elbow motif and forms, together with His101, part of a catalytic triad at the interface between three pairs of monomers [23]. Since all the known proteases have adjacent Cys/His residues, whereas a substitution by Cys/X is found in non-protease members, there is a high probability that many proteins of the adjacent group, namely those of the ThiJ/Pfpl-like proteins, also display protease activity, as most of them have a Cys/His pair in the same position. Furthermore, a consensus sequence was identified around the Cys/His pair, namely AICHGP (SEQ ID NO: 7). Proteins belonging to this group include for example the intracellular protease YhbO from E. coli, the stress protein GSP18 from B. subtilis, the chaperone Hsp31 from E. coli and the intracellular protease YDR533c from S. cerevisiae [3].

In order to classify the protease according to the invention in one of the clusters described above, computer-assisted investigations were conducted with the aid of the ExPASy website. A molecular weight of about 19.6 kDa and a theoretical pl of 5.45 (ProtParam Tool [9]) were determined. According to the SignalP 3.0 Prediction program [14] the amino acid sequence does not include a signal sequence for secretion. This points to a cytoplasmic localization, which was confirmed by the prediction program for the subcellular localization of bacterial proteins (PSORT-B[8]). A further structural comparison using the HTH prediction program (Network Protein Sequence Analysis [5]) showed that the protease according to the invention does not have any HTH-DNA-binding domains. Accordingly the protein does not appear to be a transcriptional regulator.

Based on the great similarity at the amino acid sequence level with the intracellular protease YhbO from E. coli it is more probable that the protease according to the invention from B. glumae possesses high similarity to YhbO and so belongs to the family of the ThiJ/Pfpl-like proteins. As can be seen from the alignment (FIG. 1), all conserved amino acid residues that could be identified in YhbO and other proteins of this group, are also present in the protease according to the invention. The crystal structure of YhbO was also determined and showed that the protein forms a homodimer [25]. Using native polyacrylamide gel electrophoresis, it was shown that the subunit of the protease according to the invention, which is expressed as a fusion protein in E. coli, forms a multimer conformation (data not shown).

c) Expression of the Protease According to the Invention in B. glumae

Analysis of the DNA sequence yielded two primers for amplifying the gene of the protease according to the invention directly from the genome of B. glumae PG1. The resulting PCR product was inserted in the pBBR1mcs vector with broad host specificity using the restriction sites HindIII and BamHI. The construct with the designation pBBRpro was transferred using E. coli S17-1 as donor strain and B. glumae PG1 or LU8093 as recipients. A total of four different expression studies were conducted as described above.

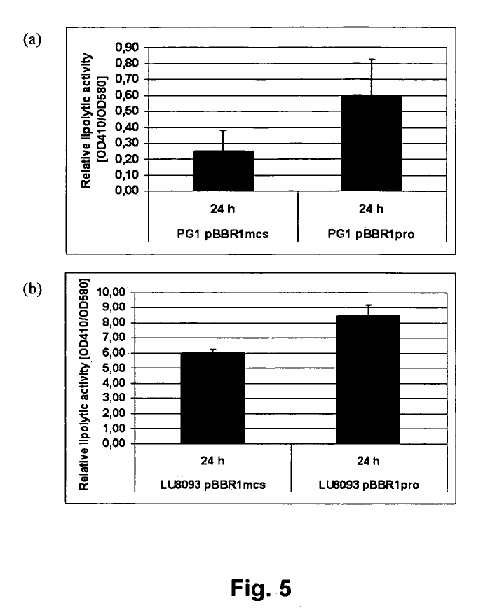

In the case of the wild-type strain B. glumae PG1 the additional expression of the plasmid pBBRpro led to a 2- to 3-fold increase in lipase activity, whereas in the production strain LU8093 a 0.3-fold increase was observed (cf. FIGS. 5a and b). The reason for this difference is that the production strain already produces an approx. 10-times higher amount of lipase than the wild-type strain, so that expression of the plasmid can no longer give rise to lipase production to the same extent as is observed in the wild-type strain.

If colonies from the expression cultures are grown on skimmed-milk agar plates, no differences are observed in halo formation (data not shown). As the protease according to the invention is presumably localized in the cytoplasm and the vector pBBR1mcs has a relatively small number of copies, simple plate cultures on indicator plates are perhaps not sufficient for detection of proteolytic activity.

d) Overexpression of the Protease According to the Invention in E. coli

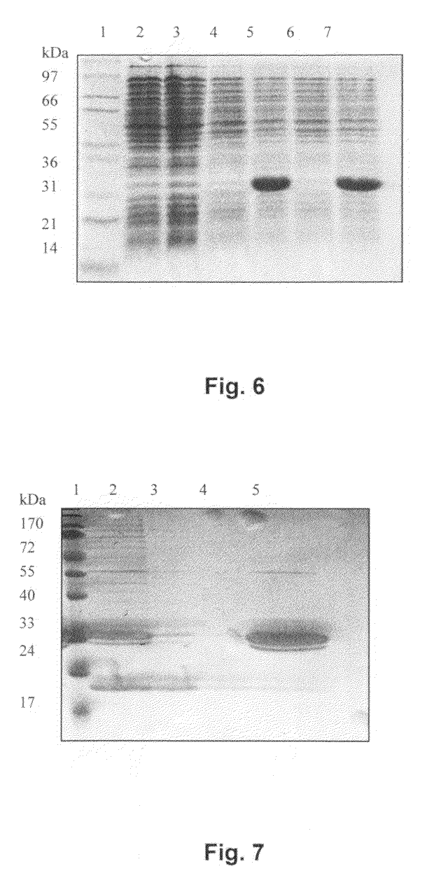

For the overexpression of the protease according to the invention, the gene was cloned in the vector pET22b under the control of the promoter T7 and the resulting construct was transformed in E. coli BL21 DE3. Preliminary overexpression investigations were conducted on a small scale with 25-ml cultures, as described above, and samples were analyzed by SDS-PAGE (cf. FIG. 6). Successful overexpression of the protein was detected 2 to 6 hours after induction. On the basis of the protein standard used, the protease according to the invention has a molecular weight of about 21 kDa, which is slightly above the calculated molecular weight of about 19.6 kDa. When colonies that have been obtained from expression cultures are grown on skimmed-milk agar plates, no differences are observed in halo formation (data not shown). As it had been shown that overexpression of the protease according to the invention does not lead to formation of inclusion bodies, it is assumed that the protein is inactive on expression in E. coli, or that skimmed milk is not a suitable substrate.

e) Purification of the Protease According to the Invention

The overexpressed protein was purified by nickel-nitrilotriacetic acid metal-affinity chromatography. 6-Liter cultures of E. coli BL21DE3 pETpro were grown overnight at 37° C. until the optical density OD₅₈₀ was 0.6 and overexpression of the protein was induced by addition of 1 mM IPTG. After 4 hours the cells were harvested and resuspended in 25 ml of ice-cold buffer (50 mM Kpi, 10 mM imidazole, pH 8.0). The cells were disrupted by incubation with lysozyme (0.5 mg/ml, 30 minutes on ice) followed by sonication (10×30 seconds). The samples were centrifuged (14000 rev/min, 20 minutes, 4° C.) and filtered to remove cellular fragments. The clear lysate was then applied to a nickel-acetonitrile column and washed with potassium phosphate buffer (50 mM Kpi, 10 mM imidazole, pH 8.0). After elution of the recombinant protein with 50 mM Kpi buffer, containing 250 mM imidazole (pH 8.0), the corresponding fractions were combined and desalted using a Sephadex-G25 column. A volume of 50 μl was taken for each purification step for the SDS-PAGE analysis (FIG. 7). After desalting of the eluate, the total volume of the purified protein was 90 ml. After Bradford assay using bovine serum albumin as standard, a protein concentration of the sample of 3 mg/ml was determined, which corresponded to an overall yield of protein of 270 mg. This purified protein can now undergo further enzyme activity tests.

f) Determination of Protease Activity in the Microtiter Plate Test

The purified protein was tested for protease activity in an enzyme plate test (Taxaprofile E, Merlin, Bornheim-Hersel). In this test, the protein to be investigated is tested in substrates for 95 aminopeptidases and proteases, 76 glycosidases, phosphatases and esterases, as well as in 17 classical reactions at pH8.2, pH7.5, pH5.5 and pH4.0. The procedure followed was that supplied by the company Merlin. The buffer used for the purified protein served as control (10 mM Kpi buffer pH6.5). After incubation of the plates for 24 h at 30° C., a visual assessment was carried out. This test was carried out a total of three times, and unambiguously positive reactions were found with the following substrates:

Four aminopeptidase substrates: lysine-α-Na, arginine-α-Na, L-alanine-α-Na, glutamate(βNa)-OH

Two glycosidase substrates: p-nitrophenyl-alpha-L-arabinopyranoside, p-nitrophenyl-β-D-galactopyranoside.

### BIBLIOGRAPHY

[1] Altschul S. F., Gish W., Miller W., Myers E. W. & Lipman D. J. (1990) Basic local alignment search tool, J. Mol. Biol. 215: 403-410
[2] Altschul S. F., Madden T. L., Schaffer A. A., Zhang J., Zhang Z., Miller W. & Lipman D. J. (1997) Gapped BLAST and PSI-Blast: a new generation of protein database search programs. Nucleic Acids Res. 25 (17): 3389-402
[3] Bandyopadhyay S., Cookson M. R. (2004) Evolutionary and functional relationships within the DJ1 superfamily. BMC Evol Biol 4 (6):1-9
[4] Bradford M. (1976): A rapid and sensitive method for the quantitation of microgram quantities of protein utilizing the principles of protein-dye binding. Anal. Biochem. 72: 248-254
[5] Dodd I. B., Egan J. B. (1990) Helix-turn-helix DNA-binding motifs prediction: Improved detection of helix-turn-helix DNA-binding motifs in protein sequences. Nucleic Acids Res 18 (17):5019-5026
[6] Figurski D. H. & Helinski D. R. (1979), Replication of an origin-containing derivative of plasmid RK2 dependent on a plasmid function provided in trans. Proc. Natl. Acad. Sci. USA 76 (4): 1648-1652
[7] Frenken, L. (1993) Characterization, biogenesis and protein engineering. PhD Thesis, University of Utrecht[8] Gardy J. L., Spencer C., Wang K., Ester M., Tusnady G. E., Simon I., Hua S., deFays K., Lambert C., Nakai K. & Brinkman F. S. L. (2003) PSORT-B: improving protein subcellular localization prediction for Gram-negative bacteria. Nucleic Acids Research 3 (13): 3613-17
[9] Gill S. C., von Hippel P. H. (1989) Calculation of protein extinction coefficients from amino acid sequence data. Anal. Biochem. 182: 319-326
[10] Higgins D., Thompson J., Gibson T. Thompson J. D., Higgins D. G., Gibson T. J. (1994), CLUSTAL W: improving the sensitivity of progressivemultiple sequence alignment through sequence weighting position-specific gap penalties and weight matrix choice. Nucleic Acids Res. 22: 4673-4680
[11] Ivanova N., Sorokin A., Anderson I., Galleron N., Candelon B., Kapatral V., Bhattacharyya A., Reznik G., Mikhailova N., Lapidus A., Chu L., Mazur M., Goltsman E., Larsen N., D'Souza M., Walunas T., Grechkin Y., Pusch G., Haselkorn R., Fonstein M., Ehrlich S. D., Overbeek R., Kyrpides N. C. (2003) Genome sequence of Bacillus cereus and comparative analysis with Bacillus anthracis. Nature 423:87-91
[12] Kunst F., et al (1997) The complete genome sequence of the Gram-positive bacterium Bacillus subtilis. Nature 390: 249-256
[13] Laemmli U. K. (1970) Cleavage of structural proteins during the assembly of the head of bacteriophage T4, Nature 227 (259): 680-5
[14] Nielsen H., Engelbrecht J., Brunak S. & von Heijne G. (1997) Identification of prokaryotic and eukaryotic signal peptides and prediction of their cleavage sites. Protein Engineering 10:1-6
[15] Perna N. T., Plunkett G. III, Burland V., Mau B. (2001) Genome sequence of enterohaemorrhagic Escherichia coli O157:H7. Nature 409:529-533
[16] Rao M. B., Tanksale A. M., Ghatge M. S., Deshpande V. V. (1998) Molecular and biotechnological aspects of micobial proteases. Microbiol. Mol. Biol. Rev. 62: 597-635
[17] Sambrook J., Fritsch E. F., Maniatis T. (1989) Molecular cloning: a laboratory manual, 2nd ed. Cold Spring Harbor Laboratory Press, Cold Spring Harbor, N.Y.
[18] Schmid A., Dordick J. S., Hauer B., Kiener A., Wubbolts M. & Witholt B. (2001) Industrial biocatalysis today and tomorrow. Nature insight 409: 258-268
[19] Simon R., Reifer U. & Puehler A. (1983) A broad host range mobilization system for in vivo genetic engineering: Transposon mutagenesis in Gram-negative bacteria. BioTechnology 1: 784-791
[20] Staskawicz B., Dahlbeck D., Keen N. & Napoli C. (1987) Molecular characterization of cloned avirulence genes form race 0 and race 1 of Pseudomonas syringae pv. Glycinea. J. Bacteriol, 169: 5789-5794
[21] Stuer W., Jaeger K. E. & Winkler U. K. (1986) Purification of extracellular lipase from Pseudomonas aeruginosa. J. Bacteriol. 168: 1070-1074
[22] Takaki Y., Kageyama Y., Shimamura S., Suzuki H., Nishi S. (2003) The complete genome sequence of the alkaliphilic Bacillus clausii KSM-K16. Submitted to the EMBL/GenBank/DDBJ databases
[23] Trotter E. W., Kao. C. M., Berenfeld L., Botstein D., Petsko G. A., & Gray J. V. (2002) Misfolded proteins are competent to mediate a subset of the responses to heat shock in Saccharomyces cerevisiae. J. Biol. Chem. 277: 44817-44825
[24] Windgassen M. (200) Proteinsekretion in Pseudomonas aeruginosa: Untersuchung der Rolle periplasmatischer Proteasen durch lnsertionsmutagenese. Diplomarbeit, Ruhr-Universitat Bochum
[25] Xinlin D., In-Geol C., Rosalind W., Weiru W., Jaru J., Hisao Y., Sung-Hou K. (2000) Crystal structure of an intracellular protease from Pyrococcus horikoshii at 2-Å resolution. PNAS 97 (26): 14079-14084
[26] Dodd I. B., Egan J. B. (1990) Helix-turn-helix DNA-binding motifs prediction: Improved detection of helix-turn-helix DNA-binding motifs in protein sequences. Nucleic Acids Res 18 (17): 5019-5026
[27] Breuer, M., Ditrich, K., Habicher, T., Hauer, B., Keβeler, M., Stürmer, R. and Zelinski, T. (2004) Industrial methods for the production of optically active intermediates. Angew. Chem. Int. Ed. 43: 788-824
[28] Jaeger, K.-E.& Eggert, T. (2002) Lipases for biotechnology. Curr. Opin. Biotechnol. 13: 390-397
[29] Schmid A., Dordick J. S., Hauer B., Kiener A., Wubbolts M. & Witholt B. (2001) Industrial biocatalysis today and tomorrow. Nature insight 409: 258-268
[30] Kovach, M. E. et al. Phillips, R. W., Elzer, P. H., Roop, R. M. and Peterson, K. M. (1994) pBBR1MCS: a broad-host-range cloning vector. BioTechniques 16 (5), 800-802
[31] Winkler, U. K. & Stuckmann, M.: Glycogen, hyaloronate, and some other polysaccharides greatly enhance the formation of exolipase by Serratiamarcescens. J. Bacteriol. 1978; 138: 663-670
[32] Hanahan D.: Studies on transformation of Escherichia coli with plasmids. Mol. Biol. 1983; 166:557-80

1. A protein comprising an amino acid sequence according to SEQ ID NO: 2; or an amino acid sequence derived from it, with at least 80% sequence homology to SEQ ID NO: 2.

2. The protein as claimed in claim 1, wherein the protein has a molecular weight of about 20-22 kDa, determined by SDS-PAGE under denaturing conditions.

3. The protein as claimed in claim 1, with a pI value in the range from 5.4 to 5.5 and/or protease activity.

4. The protein as claimed in claim 1, obtainable from bacteria of the genus Burkholderia.

5. The protein as claimed in claim 4, obtainable from Burkholderia glumae.

6. The protein as claimed in claim 1, which after expression in an extracellular lipase-producing bacterial host, increases the extracellular lipolytic activity, determined in standard conditions.

7. The protein as claimed in claim 6, wherein the host is B. glumae.

8. The protein as claimed in claim 6, wherein the extracellular lipolytic activity is increased by at least about 5% in comparison with the baseline value.

9. The protein as claimed in claim 1, encoded by a nucleic acid, comprising SEQ ID NO: 1 or a sequence derived from it with at least 80% sequence homology to SEQ ID NO: 1.

10. The nucleic acid sequence as defined in claim 9.

11. An expression construct, comprising, under the genetic control of at least one regulatory nucleic acid sequence, a nucleic acid sequence as claimed in claim 10, coding for a protein with protease activity.

12. A recombinant microorganism, genetically modified with at least one expression construct as claimed in claim 11.

13. A method of production of a protein with protease activity as claimed in claim 1, comprising cultivating a recombinant microorganism in conditions expressing the protein and isolating the protein that forms.

14. A method of production of a lipase (E.C. 3.1.1.3), comprising causing a host that is capable of lipase-production to express a functional protein with protease activity as claimed in claim 1 and to express lipase, at the same time or at a different time, and isolating the lipase that forms.

15. The method as claimed in claim 14, wherein the host is a bacterium of the genus Burkholderia.

16. The method as claimed in claim 14, wherein the lipase comprises an amino acid sequence according to SEQ ID NO: 6 or an amino acid sequence derived from it with at least 80% sequence homology to SEQ ID NO: 6.

17. The method as claimed in claim 14, wherein the lipase is encoded by a nucleic acid sequence comprising a sequence according to SEQ ID NO: 5 or a nucleic acid sequence derived from it with at least 80% sequence homology to SEQ ID NO: 5.


SEQUENCE LISTING (21 sequences)

SEQ ID NO: 1 - DNA, 540 bp, Burkholderia glumae
atgcagcagcgtggcggcagcatgagcaagaagatcgccgtcctggccgtggacgaattcgaagacagcgaactggtcgaacccctgcgggcgctgcgtaaggccggcgccgaagtcgacgtgatcagccagcaggcgggcgaagtcaaggggttccgtcacgcggacaagggggaagccgtcaaggtggaccgtacgttcgacgaagtccgggaaggcgaattcgacgcgctgctgctgcccggcggcctgctgcgtggcgacaatcggatgctgcccgcggcccgtgaattcgtgaccgccggcaagccggtcttcgcgatctgccacggcgggtggctgctgggcagcagcggggtcatcaacggccgcaagctgaccgcctggcccagcctgcaggacgacgtgaagaatgcgggcggcgaattctacgaccaggaagtggtgcgtgacaaggaccagctgatcaccagccgtaagcccgacgacctgccggccttcaaccgggaagcgctgcggctgctgggggccggcgcgtga

SEQ ID NO: 2 - protein, 179 aa, Burkholderia glumae
MQQRGGSMSKKIAVLAVDEFEDSELVEPLRALRKAGAEVDVISQQAGEVKGFRHADKGEAVKVDRTFDEVREGEFDALLLPGGLLRGDNRMLPAAREFVTAGKPVFAICHGGWLLGSSGVINGRKLTAWPSLQDDVKNAGGEFYDQEVVRDKDQLITSRKPDDLPAFNREALRLLGAGA

SEQ ID NO: 3 - DNA, 5238 bp, Artificial Sequence
  Plasmid
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccaattcgcctcatccaatgcagcagcgtggcggcagcatgagcaagaagatcgccgtcctggccgtggacgaattcgaagacagcgaactggtcgaacccctgcgggcgctgcgtaaggccggcgccgaagtcgacgtgatcagccagcaggcgggcgaagtcaaggggttccgtcacgcggacaagggggaagccgtcaaggtggaccgtacgttcgacgaagtccgggaaggcgaattcgacgcgctgctgctgcccggcggcctgctgcgtggcgacaatcggatgctgcccgcggcccgtgaattcgtgaccgccggcaagccggtcttcgcgatctgccacggcgggtggctgctgggcagcagcggggtcatcaacggccgcaagctgaccgcctggcccagcctgcaggacgacgtgaagaatgcgggcggcgaattctacgaccaggaagtggtgcgtgacaaggaccagctgatcaccagccgtaagcccgacgacctgccggccttcaaccgggaagcgctgcggctgctgggggccggcgcgtgaaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 4 - DNA, 5907 bp, Artificial Sequence
  Plasmid
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcacgcgccggcccccagcagccgcagcgcttcccggttgaaggccggcaggtcgtcgggcttacggctggtgatcagctggtccttgtcacgcaccacttcctggtcgtagaattcgccgcccgcattcttcacgtcgtcctgcaggctgggccaggcggtcagcttgcggccgttgatgaccccgctgctgcccagcagccacccgccgtggcagatcgcgaagaccggcttgccggcggtcacgaattcacgggccgcgggcagcatccgattgtcgccacgcagcaggccgccgggcagcagcagcgcgtcgaattcgccttcccggacttcgtcgaacgtacggtccaccttgacggcttcccccttgtccgcgtgacggaaccccttgacttcgcccgcctgctggctgatcacgtcgacttcggcgccggccttacgcagcgcccgcaggggttcgaccagttcgctgtcttcgaattcgtccacggccaggacggcgatcttcttgctcatgctgccgccacgctgctgcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 5 - DNA, 1077 bp, Burkholderia glumae
atggtcagatcgatgcgttccagggtggcggcgagggcggtggcatgggcgttggcggtgatgccgctggccggcgcggccgggttgacgatggccgcgtcgcccgcggccgtcgcggcggacacctacgcggcgacgcgctatccggtgatcctcgtccacggcctcgcgggcaccgacaagttcgcgaacgtggtggactattggtacggaatccagagcgatctgcaatcgcatggcgcgaaggtgtacgtcgcgaatctctcgggattccagagcgacgacgggccgaacggccgcggcgagcagctgctcgcctacgtgaagcaggtgctcgcggccaccggcgcgaccaaggtgaacctgatcggccacagccagggcggcctgacctcgcgctacgtcgcggccgtcgcgccgcaactggtggcctcggtgacgacgatcggcacgccgcatcgcggctccgagttcgccgacttcgtgcaggacgtgctgaagaccgatccgaccgggctctcgtcgacggtgatcgccgccttcgtcaacgtgttcggcacgctcgtcagcagctcgcacaacaccgaccaggacgcgctcgcggcgctgcgcacgctcaccaccgcgcagaccgccacctacaaccggaacttcccgagcgcgggcctgggcgcgcccggttcgtgccagacgggcgccgcgaccgaaaccgtcggcggcagccagcacctgctctattcgtggggcggcaccgcgatccagcccacctccaccgtgctcggcgtgaccggcgcgaccgacaccagcaccggcacgctcgacgtcgcgaacgtgaccgacccgtccacgctcgcgctgctcgccaccggcgcggtgatgatcaatcgcgcctcggggcagaacgacgggctcgtctcgcgctgcagctcgctgttcgggcaggtgatcagcaccagctaccactggaaccatctcgacgagatcaaccagctgctcggcgtgcgcggcgccaacgcggaagatccggtcgcggtgatccgcacgcacgtgaaccggctcaagctgcagggcgtgtga

SEQ ID NO: 6 - protein, 358 aa, Burkholderia glumae
MVRSMRSRVAARAVAWALAVMPLAGAAGLTMAASPAAVAADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 7 - protein, 6 aa, Burkholderia glumae
AICHGP

SEQ ID NO: 8 - DNA, 29 bp, Artificial Sequence
  PCR Primer
ctaggatccaattcgcctcatccaatgca

SEQ ID NO: 9 - DNA, 23 bp, Artificial Sequence
  PCR Primer
cgcaagctttcacgcgccggccc

SEQ ID NO: 10 - DNA, 21 bp, Artificial Sequence
  PCR Primer
gtacatatgcagcagcgtggc

SEQ ID NO: 11 - DNA, 21 bp, Artificial Sequence
  PCR Primer
atctcgagtcacgcgccggcc

SEQ ID NO: 12 - protein, 186 aa, Escherichia coli
MGNSPHPMQQRGGSMSKKIAVLITDEFEDSEFTSPADEFRKAGHEVITIEKQAGKTVKGKKGEASVTIDKSIDEVTPAEFDALLLPGGHSPDYLRGDNRFVTFTRDFVNSGKPVFAICHGPQLLISADVIRGRKLTAVKPIIIDVKNAGAEFYDQEVVVDKDQLVTSRTPDDLPAFNREALRLLGA

SEQ ID NO: 13 - protein, 172 aa, Salmonella typhimurium
MSKKIAVLITDEFEDSEFTSPAAEFRQAGHEVITIEKEAGKTVKGKKGEASVTIDKAIDDVRPDEFDALLLPGGHSPDYLRGDSRFVDFTRDFVNSGKPVFAICHGPQLLISADVIRGRKLTAVKPIIIDVKNAGAEFYDQEVVVDKDQLVTSRTPDDLPAFNREALRLLGA

SEQ ID NO: 14 - protein, 172 aa, Salmonella typhimurium; identical to SEQ ID NO: 13

SEQ ID NO: 15 - protein, 172 aa, Bacillus clausii
MGKKVAVLITDLFEDVEYTDPKESLTEAGHELVTIEKEAGKTVTGKQGNAKVTIDKSIDDVHVDDFDALLLPGGFSPDQLRADDRFVEFTKAFAQAGKPIMAICHGPQLLINADVLKGRTVTGVKPIVPDLKNAGAIFKDEEVVVCNNNLVTSRTPDDLPAFNRESLNVLAD

SEQ ID NO: 16 - protein, 171 aa, Bacillus licheniformis
MSKKIAVVLTDYFEDSEYTEPAKAFKDAGHQLTVIEKEKGKTLKGKQGEAQVETDASIDEVSPADFDALLIPGGFSPDILRADQRFVSFAKSFMDDKKPVFAICHGPQLLITAKALDGRGATGYTSIQVDLENAGVKFKDEEVVVCQNQLVTSRTPDDIPAFTRESLKLLG

SEQ ID NO: 17 - protein, 171 aa, Bacillus halodurans
MGKKIAVVVTNLFEDSEYTEPVKAFKEAGHSIVTIEKEKGIKVKGKQGESTITIDASIDEVTEDAFDALLIPGGFSPDILRADDRFVAFAKAFADAKKTIMAICHGPQLLINAGVLKGRDVTGYKSIAVDLRNAGANFYDQEVVVCGGNLVTSRTPDDLPAFNRESLNVLT

SEQ ID NO: 18 - protein, 171 aa, Bacillus cereus
MSKKIATLITDYFEDTEYTEPAEAFKQKGYELTTIEAKKGKTVHGKQGKSEVVIDKGIDDVSPENFDALFIPGGFSPDILRADERFVRFSKSFMDTKKPVFAICHGPQLLITAQTLEGRDVTGYKSIEVDLKNAGGNFHDKEVVVCQNQLVTSRQPEDIPAFIEESLRILG

SEQ ID NO: 19 - protein, 171 aa, Bacillus cereus
MSKKIATLITDYFEDTEYTEPAEAFKQKGYELTTIEMEKGKTVHGKQGKSEVVIDKGIDDVSPENFDALFIPGGFSPDILRADERFVRFSKAFMDAEKPVFAICHGPQLLITAQTLEGRDVTGYKSIEVDLKNAGGNFHNKEVVVCQNQLVTSRQPEDIPAFIEESLRILG

SEQ ID NO: 20 - protein, 166 aa, Pyrococcus horikoshii
MKVLFLTANEFEDVELIYPYHRLKEEGHEVYIASFERGTITGKHGYSVKVDLTFDKVNPEEFDALVLPGGRAPERVRLNEKAVSIARKMFSEGKPVASICHGPQILISAGVLRGRKGTSYPGIKDDMINAGVEWVDAEVVVDGNWVSSRVPADLYAWMREFVKLLK

SEQ ID NO: 21 - protein, 166 aa, Pyrococcus furiosus
MKILFLSANEFEDVELIYPYHRLKEEGHEVYIASFEKGVITGKHGYSVKVDLTFDEVNPDEFDALVLPGGRAPERVRLNEKAVEIARKMFTEGKPVATICHGPQILISAGVLKGRKGTSYIGIRDDMINAGVEWIDREVVVDGNWVSSRHPGDLYAWMREFVKLLK